(19) United States
(12) Patent Application Publication
(10) Pub. No.: US 2006/0167305 A1
(43) Pub. Date: Jul. 27, 2006

(54) Novel (meth)acrylates and methods of producing thereof

(75) Inventor: Keiichi Yokoyama, Wakayama-shi (JP)

Correspondence Address: HAMRE, SCHUMANN, MUELLER & LARSON, P.C., P.O. BOX 2902-0902, MINNEAPOLIS, MN, 55402, US

(73) Assignee: HONSHU CHEMICAL INDUSTRY CO., LTD., Tokyo (JP)

(21) Appl. No.: 11/243,525

(22) Filed: Oct. 4, 2005

(30) Foreign Application Priority Data
Oct. 8, 2004 (JP) 2004-296381
Oct. 8, 2004 (JP) 2004-296382
Dec. 6, 2004 (JP) 2004-352865

(51) Int. Cl. C07C 69/52

(52) U.S. Cl. 560/220

(57) ABSTRACT

The invention provides a (meth)acrylate represented by the general formula (I) 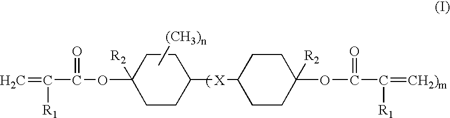
wherein X is a single bond or an alkylidene group having the general formula (II) 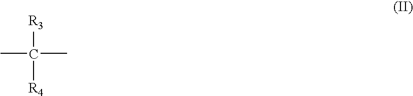
wherein R₃ and R₄ are independently hydrogen atoms or alkyl groups having 1 to 4 carbon atoms, R₁ is a hydrogen atom or a methyl group, R₂ is an alkyl group having 1 to 4 carbon atoms, m is 0 or 1, and when m is 0, n is 3, and when m is 1, n is 0. The (meth)acrylate is obtained by reacting a cycloaliphatic ketone, an organomagnesium halide and a (meth)acrylic acid ester in the presence of an amine under the industrially feasible reaction conditions and operation according to the invention.

### TECHNICAL FIELD

The present invention relates to a novel (meth)acrylate, and more particularly, to a novel mono(meth)acrylate and a di(meth)acrylate, and a method for producing thereof.

### BACKGROUND ART

Tertiary alkyl esters of (meth)acrylic acid are now in use as monomers for preparing photoresists, polymer modifiers, antistatic agents and stabilizers, and in addition, a variety of functional chemicals and raw materials therefor, however, such tertiary alkyl esters of (meth)acrylic acid that have diverse structures are still demanded so that they have much more improved functions.

Some tertiary alkyl esters of (meth)acrylic acid are already known, such as 1-ethylcyclohexyl (meth)acrylate (Japanese Patent Laid-Open No. 2000-319226), 2-methyl-2-adamantyl methacrylate (Japanese Patent Laid-Open No. 2000-229911) or 8-ethyl-8-tricyclodecanyl acrylate (Japanese Patent Laid-Open No. 2001-233832). However, in the field of monomers for preparing photoresists or polymer modifiers, development of tertiary alkyl esters of (meth)acrylic acid much improved in solubility to solvents, light permeability (transparency) or heat resistance, in particular, cycloaliphatic tertiary (meth)acrylates in which (meth)acryloyloxy groups are bonded to tertiary carbon atoms of cycloaliphatic hydrocarbon group are demanded

Various di(meth)acrylates of bisphenol compounds are likewise in use as optical materials, polymer modifiers, curable materials for dental use, antistatic agents, stabilizers and monomers for preparing photoresists, and in addition, a variety of functional chemicals and raw materials therefor, however, such di(meth)acrylates of bisphenol compounds that have diverse structures are still demanded so that they have much more improved functions.

Under these circumstances, some di(meth)acrylates of bisphenol compounds having a cyclohexylidene structure therein are proposed, such as di(meth)acrylate of 1,1-bis(4-hydroxyphenyl)cyclohexane (for example, Japanese Patent Laid-Open No. 63-215653) or di(meth)acrylate of bis(4-hydroxyl-phenyl)-3,3,5-trimethylcyclohexane (for example, Japanese Patent Laid-Open No. 07-069986). However, particularly in the field of optical materials, polymer modifiers or monomers for preparing photoresists, development of tertiary alkyl esters of (meth)acrylic acid much improved in reactivity, solubility to solvents, light permeability (transparency) or heat resistance are demanded.

On the other hand, it is already known that such a tertiary alkyl ester of (meth)acrylic acid in which a (meth)acryloyloxy group is bonded to a tertiary carbon atom of cycloaliphatic hydrocarbon group can be produced by the reaction of a tertiary alcohol with (meth) acrylic acid chloride (Experimental Chemistry Lecture 22, “Organic Synthesis IV” —Acid/Amino Acid/Peptide—, Fourth Edition, pp.50-51, published by Maruzen Co., Ltd., Nov. 30, 1992). However, in general, it is difficult from industrial point of view to apply a method in which a (meth)acrylic acid halide such as (meth)acrylic acid chloride is used to industrial use since a (meth)acrylic acid halide is chemically unstable and difficult to handle, as it generates corrosive substance if it reacts with a slight amount of water.

Therefore, for example, a method has been proposed in which 1-ethyl-1-cyclohexanol is reacted with acrylic acid in the presence of triethylamine in acetic anhydride to provide 1-ethyl-cyclohexyl acrylate (Japanese Patent Laid-Open No. 2000-319226). A further method has been proposed in which 2-adamantanone that is a cycloaliphatic ketone is used as a starting material. That is, 2-adamantanone is reacted with an alkyl metal compound and either an alkyl (meth)acrylate or anhydrous (meth)acrylic acid to provide 2-alkyl-2-adamantyl (meth)acrylate as a cycloaliphatic tertiary alkyl (meth)acrylate (Japanese Patent Laid-Open No. 2002-241342).

However, when such a method as mentioned above is employed to produce a (meth)acrylate having a bulky tertiary cycloaliphatic hydrocarbon group, there arise various problems. That is, the method needs expensive materials; the reaction steps are so complicated to be industrially employed or the reaction conditions are so severe as to be industrially employed; the reaction yield is low; and in addition, the method is accompanied by purification processes difficult to be performed to obtain high purity products on account of formation of undesired by-products and residual metals in the product.

The invention has been accomplished under these circumstances mentioned above in respect of the (meth)acrylates. It is therefore an object of the invention to provide a (meth)acrylate having improved reactivity, solubility to solvents, light permeability (transparency) or heat resistance, in particular, a 1-alkylcyclohexyl(meth)acrylate that is a tertiary alkyl ester of (meth)acrylic acid and a di(meth)acrylate that has a molecular structure in which two skeletons of 1-alkyl-cyclohexyl(meth)acrylate are bonded together.

Furthermore, the invention has been accomplished under those circumstances mentioned above in respect of the production of (meth)acrylates having a bulky tertiary cycloaliphatic hydrocarbon group. Therefore, it is a further object of the invention to provide a method which gives a desired cycloaliphatic tertiary (meth)acrylate in high yields under the reaction conditions easily employed in industrial production.

### DISCLOSURE OF THE INVENTION

The invention provides a (meth)acrylate represented by the general formula (I) 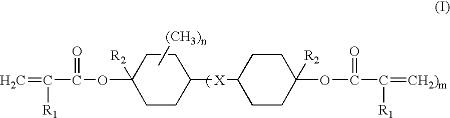
wherein X is a single bond or an alkylidene group having the general formula (II) 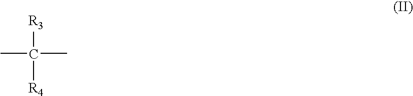
wherein R₃ and R₄ are independently hydrogen atoms or alkyl groups having 1 to 4 carbon atoms, R₁ is a hydrogen atom or a methyl group, R₂ is an alkyl group having 1 to 4 carbon atoms, m is 0 or 1, and when m is 0, n is 3, and when m is 1, n is 0.

In more detail, as one aspect of the invention, it provides a 1-alkylcyclohexyl (meth)acrylate represented by the general formula (Ia) 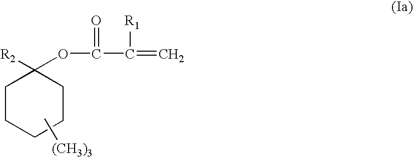
wherein R₁ is a hydrogen atom or a methyl group, and R₂ is an alkyl group having 1 to 4 carbon atoms.

As a further aspect of the invention, it provides a di(meth)acrylate represented by the general formula (Ib) 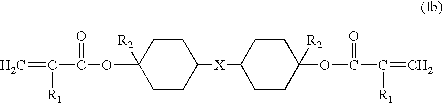
wherein X is a single bond or an alkylidene group having the general formula (II) 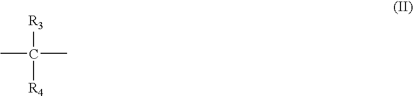
wherein R₃ and R₄ are independently hydrogen atoms or alkyl groups having 1 to 4 carbon atoms, R₁ is a hydrogen atom or a methyl group, and R₂ is an alkyl group having 1 to 4 carbon atoms.

In addition, the invention provides a method of producing a cycloaliphatic tertiary (meth)acrylate which comprises reacting a cycloaliphatic ketone with an organomagnesium halide and a (meth)acrylic acid ester represented by the general formula (III) 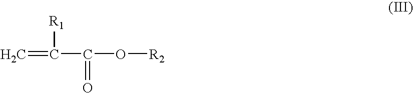
wherein R₁ is a hydrogen atom or a methyl group, and R₂ is an aryl group or a vinyl group, in the presence of an amine.

### BRIEF EXPLANATION OF THE DRAWINGS

FIG. 1 is an infrared spectroscopy of 1,3,3,5-tetramethyl-cyclohexyl methacrylate of the invention;

FIG. 2 is an infrared spectroscopy of 1,3,3,5-tetramethyl-cyclohexyl acrylate of the invention;

FIG. 3 is an infrared spectroscopy of 2,2-bis-(4-methacryloyloxy-4-methylcyclohexylpropane of the invention; and

FIG. 4 is an infrared spectroscopy of 4,4′-dimethacryloyl-oxy-4,4′-dimethylbicyclohexyl of the invention.

### BEST MODE FOR CARRYING OUT THE INVENTION

The (meth)acrylate of the invention is a mono(meth)-acrylate or a di(meth)acrylate represented by the general formula (I) 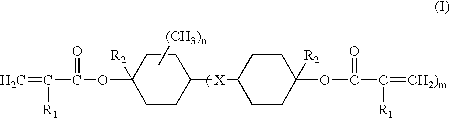
wherein X is a single bond or an alkylidene group having the general formula (II) 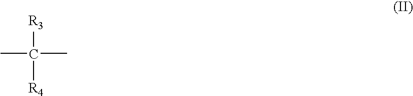
wherein R₃ and R₄ are independently hydrogen atoms or alkyl groups having 1 to 4 carbon atoms, R₁ is a hydrogen atom or a methyl group, R₂ is an alkyl group having 1 to 4 carbon atoms, m is 0 or 1, and when m is 0, n is 3, and when m is 1, n is 0. Herein the invention, (meth)acrylates means acrylates or methacrylates.

First, the mono(meth)acrylate of the invention is a 1-alkylcyclohexyl (meth)acrylate having the general formula (Ia) 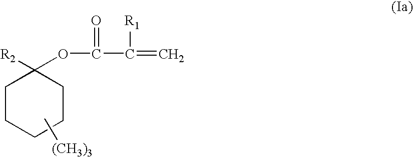
in which R₁ is a hydrogen atom or a methyl group, and R₂ is an alkyl group having 1 to 4 carbon atoms.

In the 1-alkylcyclohexyl (meth)acrylate represented by the general formula (I), R₁ is a hydrogen atom or a methyl group, and R₂ is an alkyl group having 1 to 4 carbon atoms. The alkyl groups having 1 to 4 carbon atoms include methyl, ethyl, propyl or butyl groups, and among these alkyl groups, propyl and butyl groups may be either branched or linear.

Thus, there may be mentioned as examples of the cyclohexyl (meth)acrylates of the invention:
1,2,3,5-tetramethylcyclohexyl acrylate,
1,2,3,5-tetramethylcyclohexyl methacrylate,
1,3,3,5-tetramethylcyclohexyl acrylate,
1,3,3,5-tetramethylcyclohexyl methacrylate,
1,2,3,4-tetramethylcyclohexyl acrylate,
1,2,3,4-tetramethylcyclohexyl methacrylate,
1,3,4,5-tetramethylcyclohexyl acrylate,
1,3,4,5-tetramethylcyclohexyl methacrylate,
1,3,3,4-tetramethylcyclohexyl acrylate,
1,3,3,4-tetramethylcyclohexyl methacrylate,
1-ethyl-3,3,5-trimethylcyclohexyl acrylate,
1-ethyl-3,3,5-trimethylcyclohexyl methacrylate,
1-isopropyl-3,3,5-trimethylcyclohexyl acrylate,
1-isopropyl-3,3,5-trimethylcyclohexyl methacrylate,
1-n-propyl-3,3,5-trimethylcyclohexyl acrylate,
1-n-propyl-3,3,5-trimethylcyclohexyl methacrylate,
1-n-butyl-3,3,5-trimethylcyclohexyl acrylate,
1-n-butyl-3,3,5-trimethylcyclohexyl methacrylate,
1-isobutyl-3,3,5-trimethylcyclohexyl acrylate,
1-isobutyl-3,3,5-trimethylcyclohexyl methacrylate,
1-t-butyl-3,3,5-trimethylcyclohexyl acrylate, and
1-t-butyl-3,3,5-trimethylcyclohexyl methacrylate.

These 1-alkylcyclohexyl (meth)acrylates of the invention can be obtained by, for example, reacting an alkyl Grignard reagent (V) such as methylmagnesium chloride with trimethylcyclohexanone (IV), and then reacting the resulting addition reaction product (VI) with a (meth)acryic acid halide such as (meth)acryloyl chloride (VII), as shown in the scheme below.

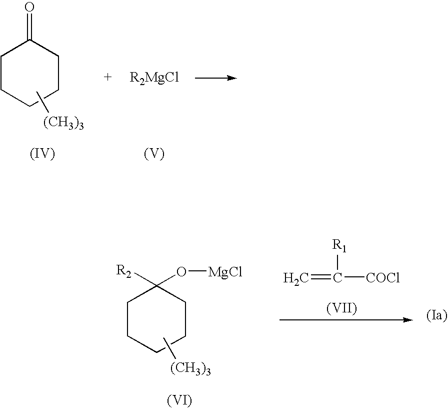

According to the invention, it is preferred that the addition reaction product (VI) of trimethylcyclohexanone with an alkyl Grignard reagent is reacted with a (meth)acrylic acid halide, as shown in the above scheme.

The trimethylcyclohexanone represented by the formula (IV) includes, for example, 2,3,5-trimethylcyclohexanone, 2,3,4-trimethylcyclohexanone, 2,3,6-trimethylcyclohexanone, 3,3,5-trimethylcyclohexanone, 3,4,6-trimethylcyclohexanone, 3,5,6-trimethylcyclohexanone, and the like, among which 3,3,5-trimethylcyclohexanone is particularly preferred.

On the other hand, the Grignard reagent used includes, for example, methylmagnesium chloride, ethylmagnesium chloride, and methylmagnesium bromide. The reaction of the trimethylcyclohexanone and the alkyl Grignard reagent is carried out in a reaction solvent. Anhydrous tetrahydrofuran is preferably used as a solvent.

After the Grignard reaction, the resulting addition reaction product is reacted with (meth)acryloyl chloride in a conventional method, as mentioned above, thereby providing the desired novel 1-alkylcyclohexyl (meth)acrylate of the invention. The desired high purity product can be obtained as follows, for example. After the reaction, the resulting reaction mixture is neutralized with an aqueous alkaline solution such as a saturated aqueous solution of ammonium chloride, and the desired product is extracted with a solvent such as ethyl acetate or diethyl ether, followed by washing and drying the product.

The 1-alkylcyclohexyl (meth)acrylate of the invention falls under the tertiary alkyl esters of (meth)acrylic acid and is superior in solubility to solvents, light permeability (transparency) and heat resistance among others, so that it is useful in the field of monomers for preparation of photoresists or polymer modifiers.

Second, the di(meth)acrylate of the invention is represented by the general formula (Ib) 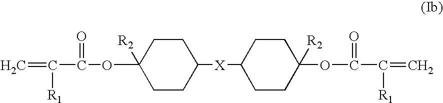
in which X is a single bond or an alkylidene group having the general formula (II) 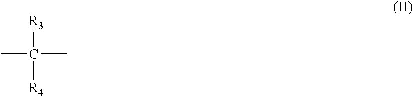
wherein R₃ and R₄ are independently hydrogen atoms or alkyl groups having 1 to 4 carbon atoms, R₁ is a hydrogen atom or a methyl group, and R₂ is an alkyl group having 1 to 4 carbon atoms.

In the di(meth)acrylate represented by the general formula (Ib), R₁ is a hydrogen atom or a methyl group, and R₂ is an alkyl group having 1 to 4 carbon atoms. The alkyl group having 1 to 4 carbon atoms includes methyl, ethyl, propyl or butyl groups, and among these alkyl groups, propyl and butyl groups may be either branched or linear.

In the alkylidene group presented by the general formula (II), R₃ and R₄ are independently hydrogen atoms or alkyl groups having 1 to 4 carbon atoms. The alkyl group having 1 to 4 carbon atoms includes methyl, ethyl, propyl or butyl groups, and among these alkyl groups, propyl and butyl groups may be either branched or linear.

Thus, there are mentioned as examples of the di(meth)acrylate of the invention when X is a single bond:
4,4′-di(meth)acryloyloxy-4,4′-dimethylbicyclohexyl,
4,4′-di(meth)acryloyloxy-4,4′-diethylbicyclohexyl,
4,4′-di(meth)acryloyloxy-4,4′-di-n-propylbicyclohexyl,
4,440 -di(meth)acryloyloxy-4,4′-diisopropylbicyclohexyl,
4,4′-di(meth)acryloyloxy-4,4′-di-n-butylbicyclohexyl, and
4,4′-di(meth)acryloyloxy-4,4′-diisobutylbicyclohexyl.

In turn, there are mentioned as examples of the di(meth)acrylate of the invention when X is an alkylidene group:
bis(4-(meth)acryloyloxy-4-methylcyclohexyl)methane,
bis(4-(meth)acryloyloxy-4-ethylcyclohexyl)methane,
bis(4-(meth)acryloyloxy-4-n-propylcyclohexyl)methane,
bis(4-(meth)acryloyloxy-4-isopropylcyclohexyl)methane,
bis(4-(meth)acryloyloxy-4-n-butylcyclohexyl) methane,
bis(4-(meth)acryloyloxy-4-isobutyl cyclohexyl) methane,
1,1-bis(4-(meth)acryloyloxy-4-methylcyclohexyl)ethane,
1,1-bis(4-(meth)acryloyloxy-4-methylcyclohexyl)propane,
1,1-bis(4-(meth)acryloyloxy-4-methylcyclohexyl)butane,
2-methyl-1,1-bis(4-(meth)acryloyloxy-4-methylcyclohexyl)-propane,
3-methyl-1,1-bis(4-(meth)acryloyloxy-4-methylcyclohexyl)-butane,
2,2-bis(4-(meth)acryloyloxy-4-methylcyclohexyl)propane,
2,2-bis(4-(meth)acryloyloxy-4-methylcyclohexyl)butane,
4-methyl-2,2-bis(4-(meth)acryloyloxy-4-methylcyclohexyl)-pentane, and
3,3bis(4-(meth)acryloyloxy-4-methylcyclohexyl)pentane.

These di(meth)acrylates of the invention can be obtained, for example, by reacting a biscyclohexanone (VIII) with an alkyl Grignard reagent (V) such as methylmagnesium chloride, and then reacting the resulting addition reaction product (IX) with a (meth)acryic acid halide such as (meth)acryloyl chloride (VII), as shown in the scheme below.

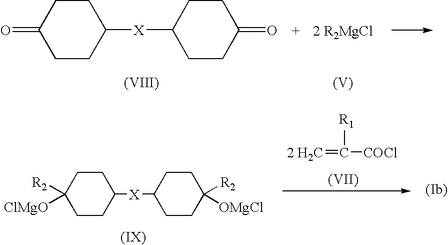

According to the invention, it is preferred that the addition reaction product (IX) of the biscyclohexanone with the alkyl Grignard reagent is reacted with a (meth)acrylic acid halide, as shown in the above scheme.

The biscyclohexanone used includes, for example, 1,1′-bicyclohexyl-4,4′-dione, 4,4′-methylenebiscyclohexane-1-one, 4,4′-ethylidenebiscyclohexane-1-one, 4,4′-propylidenebis-cyclohexane-1-one, 4,4′-butylidenebiscyclohexane-1-one, 4,4′-(2-methylpropylidene)biscyclohexane-1-one, 4,4′-(3-methyl-butylidene)biscyclohexane-1-one, 4,4′-(1-methylethylidene)-biscyclohexane-1-one, 4,4′-(1-methylpropylidene)biscyclohexane-1-one, 4,4′-(1,3-dimethylbutylidene)biscyclohexane-1-one, and 4,4′-(1-ethylpropylidene)biscyclohexane-1-one. Among these biscyclohexanones are particularly preferred 1,1′-bicyclohexyl-4,4′-dione, 4,4′-methylenebiscyclohexane-1-one or 4,4′-(1-methyl-ethylidene)biscyclohexane-1-one according to the invention.

On the other hand, likewise in the production of 1-alkylcyclohexyl(meth)acrylates, the Grignard reagent used includes, for example, methylmagnesium chloride, ethylmagnesium chloride, and methylmagnesium bromide. The reaction of the biscyclohexanone and the alkyl Grignard reagent is carried out in a reaction solvent. Anhydrous tetrahydrofuran is used as a solvent, for example.

After the Grignard reaction, the resulting addition reaction product is reacted with a (meth)acryloyl chloride in a conventional method, as mentioned above, thereby providing the desired novel di(meth)acrylate of the invention.

The high purity product of the desired compound can be obtained as follows, for example. After the reaction, the resulting reaction mixture is neutralized with an aqueous alkaline solution such as a saturated aqueous solution of ammonium chloride, and the desired product is extracted by using a solvent such as ethyl acetate or diethyl ether, followed by washing and drying the product.

The di(meth)acrylate of the invention is such that it has a tertiary alkyl group in the molecule that is derived from a biscyclohexyl skeleton or a bicyclihexyl skeleton, and it is superior in reactivity, solubility to solvents, light permeability (transparency) and heat resistance among others, so that it is useful in the field of optical materials, monomers for preparation of photoresists or polymer modifiers.

As described above, the (meth)acrylate of the invention can be obtained by reacting a ketone with a Grignard reagent, and then reacting the resulting addition reaction product with a (meth)acrylic acid halide such as (meth)acrylic acid chloride. However, it can be also obtained by a method in which a (meth)acrylic acid halide is not used so that it is industrially advantageous.

Thus, the invention provides a method of producing a cycloaliphatic tertiary (meth)acrylate which comprises reacting a cycloaliphatic ketone, an organomagnesium halide and a (meth)acrylic acid ester selected from the group consisting of an aryl (meth)acrylate and vinyl (meth)acrylate in the presence of an amine.

In the invention, the cycloaliphatic ketone used includes cycloaliphatic monoketones, cycloaliphatic diketones and biscycloaliphatic diketones. More specifically, the cycloaliphatic ketone includes, for example, cyclopentanone, 9-fluorenone, cyclohexanone, cyclooctanone and adamantanone which may have substituents thereon. In particular, the cyclohexanone having substituents thereon is represented by the general formula (X) 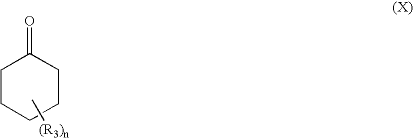
in which R₃ is a hydrocarbon group and n is an integer of 1 to 3. The group R₃ is independently an alkyl group, and it is preferably independently an alkyl group of 1 to 4 carbon atoms, and most preferably it is a methyl group. Accordingly, preferred examples of the cyclohexanone which has the general formula (X) and substituents include, for example, 3-methylcyclohexanone, 2,5-dimethylcyclohexanone, 3,3,5-trimethylcyclohexanone and 2,3,5-trimethylcyclohexanone. On the other hand, there may be mentioned cyclohexane-1,4-dione as an example of cycloaliphatic diketones.

The biscycloaliphatic diketone used includes those represented by the general formula (XI) 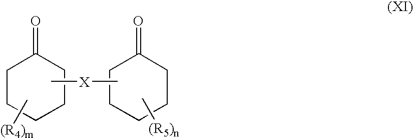
in which X is a single bond, an alkylene group or a cycloalkylene group, R₄ and R₅ are hydrocarbon groups, and m and n are independently integers of 0 to 3.

The groups R₄ and R₅ are independently alkyl groups, preferably independently alkyl groups of 1 to 4 carbon atoms, and most preferably independently methyl groups or ethyl groups. The alkylene group is preferably those of 1 to 10 carbon atoms, with preferred examples being, for example, methylene, ethylene, ethylidene, propylene, propylidene group, etc. The cycloalkylene group is preferably such that it has 5 or 6 carbon atoms, with preferred examples being 1,4-cyclohexylene or cyclohexylidene groups.

Accordingly, preferred examples of biscycloaliphatic diketone used include, for example, bi(4-oxocyclohexyl), bi(3-methyl-4-oxocyclohexyl), bi(3,5-dimethyl-4-oxocyclohexyl), bis(4-oxocyclohexyl)methane, bis(2-oxocyclohexyl)methane, bis(2-ethyl-4-oxocyclohexyl)methane, (2-oxocyclohexyl)-(4-oxo-cyclohexyl)methane and 2,2-bis(4-oxocyclohexyl)propane.

The organomagnesium halide used is represented by the general formula (XII)
R₆MgX (XII)
in which R₆ is a hydrocarbon group and X is a halogen atom. In the organomagnesium halide represented by the general formula (XII), the hydrocarbon group R₆ includes, for example, an alkyl group such as methyl, ethyl, propyl or butyl group, a cycloalkyl group such as cyclopentyl or cyclohexyl group, or an aryl group such as a phenyl group, and preferably an alkyl or an aryl group, and more preferably an alkyl group of 1 to 4 carbon atoms or a phenyl group. On the other hand, preferred halogen atoms are chlorine or bromine atoms.

Accordingly, preferred examples of the organomagnesium halide used includes, for example, methylmagnesium chloride, methylmagnesium bromide, ethylmagnesium chloride, n-propyl-magnesium chloride, isobutylmagnesium chloride and phenylmagnesium bromide.

The organomagnesium halide is used usually as a solution prepared by dissolving it in a solvent. The solvent used includes, for example, ethers such as tetrahydrofuran or diethyl ether, hydrocarbons such as hexane, heptane, cyclohexane, benzene or toluene, halogen compounds such as carbon tetrachloride or dichloromethane. However, the solvent used is not specifically limited to those exemplified above, but any solvent inactive in the reaction may be used. The solution of organomagnesium halide is used in an amount of 1 to 10 equivalents, preferably 1 to 2 equivalents, in relation to a cycloaliphatic ketone used.

According to the method of the invention, the reaction of the above-mentioned cycloaliphatic ketone, the organomagnesium halide and the (meth)acrylic acid ester represented by the general formula (III) 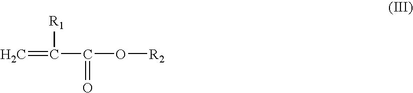
wherein R₁ is a hydrogen atom or a methyl group, and R₂ is an aryl group or a vinyl group provides the desired cycloaliphatic tertiary (meth)acrylate.

In the (meth)acrylic acid ester represented by the general formula (III), the group R₁ is a hydrogen atom or a methyl group, and R₂ is an aryl group or a vinyl group. The aryl group includes mono- and polynuclear aromatic groups such as a phenyl group, a biphenyl group, a naphthyl group, an anthryl group or a phenanthryl group, which may have substituents thereon. The aryl group further includes hetero-atom containing aromatic groups such as a 1,3-imidazolyl group or a furyl group.

According to the invention, however, vinyl (meth)acrylate or phenyl (meth)acrylate is preferably used as the above-mentioned (meth)acrylic acid ester. Such a (meth)acrylic acid ester is used usually in an amount of 1 to 100 equivalents, preferably 1 to 20 equivalents, and more preferably 1 to 5 equivalents, in relation to a cycloaliphatic ketone used.

In the method of the invention, the cycloaliphatic ketone, the organomagnesium halide and the (meth)acrylic acid ester are reacted in the presence of an amine. The amine activates the cycloaliphatic tertiary magnesium halide alkoxide produced by the reaction of the cycloaliphatic ketone and the organomagnesium halide so that it carries out a role to promote the transesterification of the (meth)acrylic acid ester. Various primary, secondary or tertiary amines are used as the amine.

For example, the primary amine usable includes methylamine, ethylamine, n-propylamine, ethylene diamine, tetramethylene diamine, allylamine, cyclohexylamine and benzylamine; the secondary amine usable includes dimethylamine, diethylamine, diphenylamine, N-methylaniline and 2,2,6,6-tetramethylpiperidine; and the teritiary amine usable includes trimethylamine, triethylamine, tributylamine, N,N,N′,N′-tetramethylethylene diamine, tribenzylamine and 4-(N,N-dimethyl)aminopyridine. Among these amines, tertiary amines are preferred, in particular, such as N,N,N′,N′-tetramethylethylene diamine.

According to the invention, a base such as an alkoxide (e.g., sodium methoxide) or a quaternary ammonium hydroxide (e.g., tetramethylammonium hydroxide) may be used in combination with the amine in the reaction. In the invention, the amine is used usually in the range of 1 to 40 equivalents, preferably 2 to 9 equivalents, in relation to a cycloaliphatic ketone used.

A preferred method of the invention to produce a cycloaliphatic tertiary (meth)acrylate is as follows. First, a cycloaliphatic ketone and an organomagnesium halide are reacted together in a solvent to generate a tertiary cycloaliphatic magnesium halide alkoxide. Then, either after the addition of (meth)acrylic acid ester, an amine is added to the resulting reaction mixture, or both the (meth)acrylic acid ester and an amine are added simultaneously to the resulting reaction mixture, or after the addition of an amine, the (meth)acrylic acid ester is added to the resulting reaction mixture, so as to react the tertiary cycloaliphatic magnesium halide alkoxide with the (meth)acrylic acid ester in the presence of an amine. After the reaction, the desired cycloaliphatic tertiary (meth)acrylate is separated from the resulting reaction mixture.

When a cycloaliphatic ketone, an organomagnesium halide and a (meth)acrylic acid ester are subjected to the reaction, the order of the reaction to be carried out is not specifically limited in the invention. However, according to a preferred embodiment of the invention, a solution of an organomagnesium halide is placed in a reaction vessel, into which a cycloaliphatic ketone is added so that the cycloaliphatic ketone is reacted with the organomagnesium halide so as to generate a tertiary cycloaliphatic magnesium halide alkoxide in the reaction mixture. Then, as mentioned above, either after the addition of (meth)acrylic acid ester, the amine is added to the reaction mixture, or both the (meth)acrylic acid ester and the amine are added to the reaction mixture simultaneously, so that the resulting tertiary cycloaliphatic magnesium halide alkoxide and the (meth)acrylic acid ester are reacted in the presence of the amine, thereby providing the desired cycloaliphatic tertiary (meth)acrylate. In the method as mentioned above, the amine may be added to the reaction mixture before the addition of the (meth)acrylic acid ester.

However, when a cycloaliphatic ketone and an organomagnesium halide are reacted, the order of the reaction to be carried out is not limited specifically to the exemplified above. For example, a solution of a cycloaliphatic ketone is first placed in a reaction vessel, and then a solution of an organomagnesium halide is added into the reaction vessel so that the reaction between the cycloaliphatic ketone and the organomagnesium halide takes place to generate a tertiary cycloaliphatic magnesium halide alkoxide. Then, either after the addition of (meth)acrylic acid ester, the amine is added to the reaction mixture, or both the (meth)acrylic acid ester and the amine are added to the reaction mixture simultaneously. Also in this case, the amine may be added to the reaction mixture before the addition of the (meth)acrylic acid ester.

The reaction is shown in a scheme below taking the case of 3,3,5-trimethylcyclohexanone is used as a cycloaliphatic ketone. As shown, 3,3,5-trimethylcyclohexanone (1) is reacted with an alkylmagnesium chloride to obtain a tertiary cycloaliphatic magnesium halide alkoxide (2) which has as substituent an alkyl group R₆ derived from the alkylmagnesium chloride. Then, the tertiary cycloaliphatic magnesium halide alkoxide is reacted with the (meth)acrylic acid ester in the presence of the amine, thereby providing the desired cycloaliphatic tertiary (meth)acrylate (3).

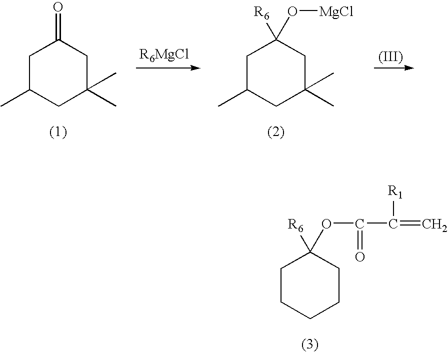

Thus, according to the invention, a desired mono(meth)acrylate can be obtained from a cycloaliphatic monoketone, and in the same manner, a corresponding di(meth)acrylate can be obtained from a cycloaliphatic diketone or a biscycloaliphatic diketone.

Among the amines mentioned above, the primary and secondary amines usually react with an organomagnesium halide. Accordingly, it is preferred that these amines are added to the reaction mixture after the reaction of cycloaliphatic ketone with organomagnesium halide. On the other hand, when a tertiary amine is used, there is no need of considering such limitation. Therefore, for example, the tertiary amine may be added to the reaction mixture at any stage of the reaction, either before or after the reaction of the cycloaliphatic ketone with the organomagnesium halide. However, it is usually preferred that the tertiary amine is added to the reaction mixture after the reaction of the cycloaliphatic ketone with the organomagnesium halide.

In general, as mentioned above, the reaction of a cycloaliphatic ketone and an organomagnesium halide generates a tertiary cycloaliphatic magnesium halide alkoxide, which has low reactivity in respect of esterification, and that is why the expected transesterification reaction hardly progresses even if the reaction is carried out in the presence of a reaction promoter such as an amine when an alkyl ester of (meth)acrylic acid such as methyl (meth)acrylate conventionally known as an esterification agent is used. However, according to the invention, the reaction of the above-mentioned (meth)acrylic acid ester with the tertiary cycloaliphatic magnesium halide alkoxide in the presence of the amine provides the desired tertiary (meth)acrylate in high yields.

The reaction temperature at which a cycloaliphatic ketone and an organomagnesium halide are reacted is usually in the range of −70° C. to 200° C., preferably in the range of −50° C. to 100° C. The reaction temperature at which a tertiary cycloaliphatic magnesium halide alkoxide and the (meth)acrylic acid ester after the above-mentioned reaction may be in the same range as above, but it is not necessary that the reaction temperatures are the same. The reaction time for which a cycloaliphatic ketone and an organomagnesium halide are reacted is usually in the range of 0.2 hours to 500 hours. The reaction time at which a cycloaliphatic magnesium halide alkoxide and the (meth)acrylic acid ester after the above-mentioned reaction is usually in the range of 0.5 hours to 100 hours.

According to the method of the invention, a high purity product of the desired cycloaliphatic tertiary (meth)acrylate can be obtained by a conventionally known after-treatment and purification process of the reaction mixture obtained in the reaction. By way of example, the obtained reaction mixture is concentrated under reduced pressure, the resulting concentrate is either washed or extracted with an appropriate organic solvent, followed by washing with water, and then if necessary, the product is subjected to, for example, distillation purification, column purification, or recrystallization.

The method of the invention as described above provides a variety of cycloaliphatic tertiary (meth)acrylates depending on the cycloaliphatic ketone, (meth)acrylic acid ester and organomagnesium halide used. For instance, the use of cyclohexanone, methacrylic acid ester and isopropyl magnesium halide provides 1-ethylcyclohexyl (meth)acrylate; the use of 3,3,5-trimethylcyclohexanone, methacrylic acid ester and methyl magnesium halide provides 1,3,3,5-tetramethylcyclohexyl methacrylate; the use of 3,3,5-trimethylcyclohexanone, acrylic acid ester and methylmagnesium halide provides 1,3,3,5-tetramethylcyclohexyl acrylate; the use of 4,4′-oxybicyclohexane, methacrylic acid ester and methylmagnesium halide provides 4,4′-di(methacryloyloxy)-4,4′-dimethylbicyclohexyl; and the use of 2,2-bis(4-oxycyclohexyl)propane, methacrylic acid ester and methylmagnesium halide provides 2,2-bis(4-methacryloyloxy-4-methylcyclohexyl)propane.

According to the method of the invention, a high purity product of the desired cycloaliphatic tertiary (meth)acrylate can be obtained in high yields by using a cycloaliphatic ketone, an organomagnesium halide and a (meth)acrylic acid ester as defined hereinabove as staring materials and reacting them under the industrially feasible reaction conditions and operation.

### EXAMPLES

The invention is explained with reference to examples, however, the invention is not limited thereto.

Examples of Mono(meth)acrylates

### Example 1

### Synthesis of 1,3,3,5-tetramethylcyclohexyl Methacrylate

50 mL of anhydrous tetrahydrofuran was placed in a reaction vessel under an atmosphere of argon, into which 12.5 mL of tetrahydrofuran solution of 3.0 mol/L concentration of methylmagnesium chloride was added with stirring. Under an atmosphere of argon, 5.0 g of 3,3,5-trimethylcyclohexanone was added dropwise from a dropping funnel to the tetrahydrofuran solution of methylmagnesium chloride while the resulting reaction mixture was stirred and kept at a temperature of 50° C. or less. Then, the dropping funnel was washed with 25 mL of anhydrous tetrahydrofuran and the wash was also added dropwise from the dropping funnel to the reaction mixture. Thereafter, the reaction mixture was stirred for one hour.

Subsequently, 5.12 g of methacryloyl chloride was added dropwise to the reaction mixture with stirring under an atmosphere of argon and thereafter the reaction mixture was stirred for three hours at room temperature. The resulting reaction mixture was poured into a saturated aqueous solution of ammonium chloride, followed by extracting with ethyl acetate. The thus obtained organic layer was washed with a saturated aqueous solution of sodium bicarbonate and water in this order, and was then subjected to concentration under reduced pressure. The thus obtained oily material was purified by silica gel column chromatography (using an eluate of 10% ethyl acetate/hexane), thereby providing 4.9 g of 1,3,3,5-tetramethylcyclohexyl methacrylate as colorless oil. The yield was found to be 61 mol %.

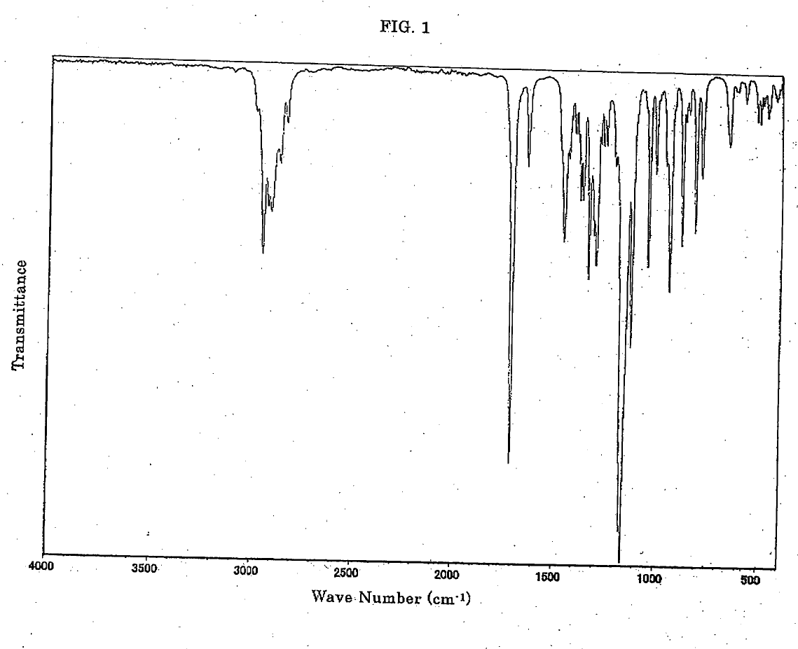

Infrared spectrum: As shown in FIG. 1, an absorption peak of vC=O was observed at 1710 cm⁻¹.

Proton nuclear magnetic resonance spectra (solvent: CDCl₃, 400 MHz):

TABLE 1

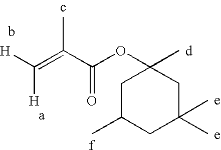

| Assignment | δ (ppm) | Signal | Number of Protons |
|---|---|---|---|
| a | 6.00 | s | 1 |
| b | 5.45 | s | 1 |
| c | 1.88 | s | 3 |
| d | 1.45 | s | 3 |
| e | 0.93 | s | 3 |
| e | 0.88 | s | 3 |
| f | 0.89 | d | 3 |

### Example 2

### Synthesis of 1,3,3,5-tetramethylcyclohexyl Acrylate

3.74 g of acryloyl chloride was used in place of 5.12 g of methacryloyl chloride in Example 1, and otherwise in the same manner as in Example 1, 3.2 g of 1,3,3,5-tetramethylcyclohexyl acrylate was obtained as colorless oil. The yield was found to be 42 mol %.

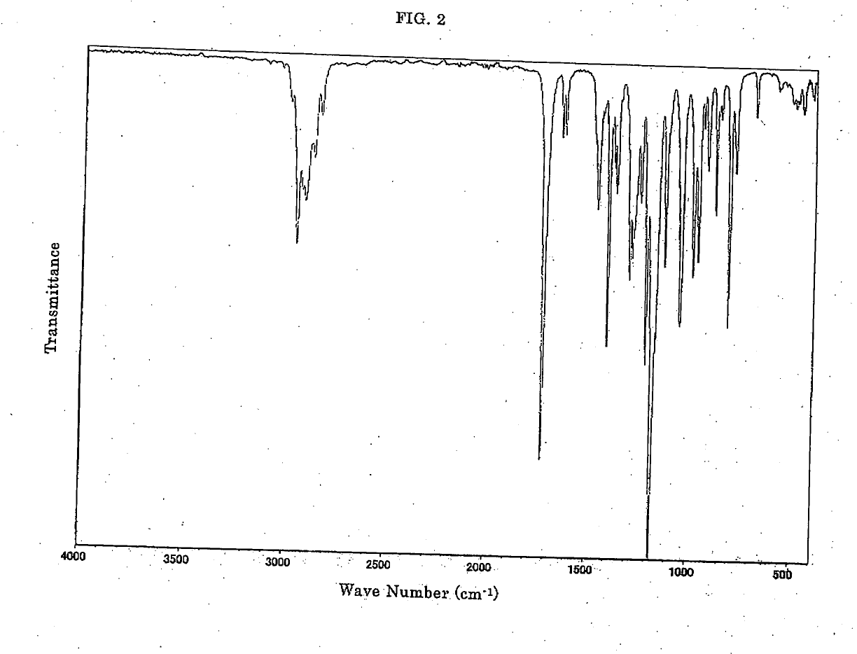

Infrared spectroscopy: As shown in FIG. 2, an absorption peak of vC=O was observed at 1720 cm⁻¹.

Proton nuclear magnetic resonance spectra (solvent: CDCl₃, 400 MHz):

TABLE 2

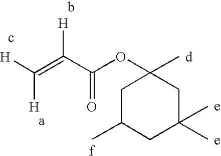

| Assignment | δ (ppm) | Signal | Number of Protons |
|---|---|---|---|
| a | 6.25 | d | 1 |
| b | 6.02 | m | 1 |
| c | 5.70 | d | 1 |
| d | 1.45 | s | 3 |
| e | 0.93 | s | 3 |
| e | 0.88 | s | 3 |
| f | 0.89 | d | 3 |

Examples of Di(meth)acrylates

### Example 3

### Synthesis of 2,2-bis(4-methacryloyloxy-4-methylcyclohexyl)-propane

50 mL of anhydrous tetrahydrofuran was placed in a reaction vessel under an atmosphere of argon, into which 14.8 mL of tetrahydrofuran solution of 3.0 mol/L concentration of methylmagnesium chloride was added with stirring. Under an atmosphere of argon, a suspension of 5.0 g of 2,2-bis(4-oxocyclohexyl)propane in 150 mL of tetrahydrofuran was added dropwise from a dropping funnel to the tetrahydrofuran solution of methylmagnesium chloride while the resulting reaction mixture was stirred and kept at a temperature of 50° C. or less. Then, another 100 mL of anhydrous tetrahydrofuran was added and the reaction mixture was stirred for one hour.

Subsequently, 6.08 g of methacryloyl chloride was added dropwise to the reaction mixture with stirring under an atmosphere of argon, and then the reaction mixture was stirred for 17 hours at room temperature. The resulting reaction mixture was poured into a saturated aqueous solution of ammonium chloride, followed by extracting with ethyl acetate. The thus obtained organic layer was washed with saturated aqueous solution of sodium bicarbonate and water in this order, and was then subjected to concentration under reduced pressure. The thus obtained oily material was purified by silica gel column chromatography (using an eluate of 5% ethyl acetate/hexane), thereby providing 4.0 g of 2,2-bis(4-methacryloyloxy-4-methyl-cyclohexyl)propane as colorless oil. The yield was found to be 47 mol %.

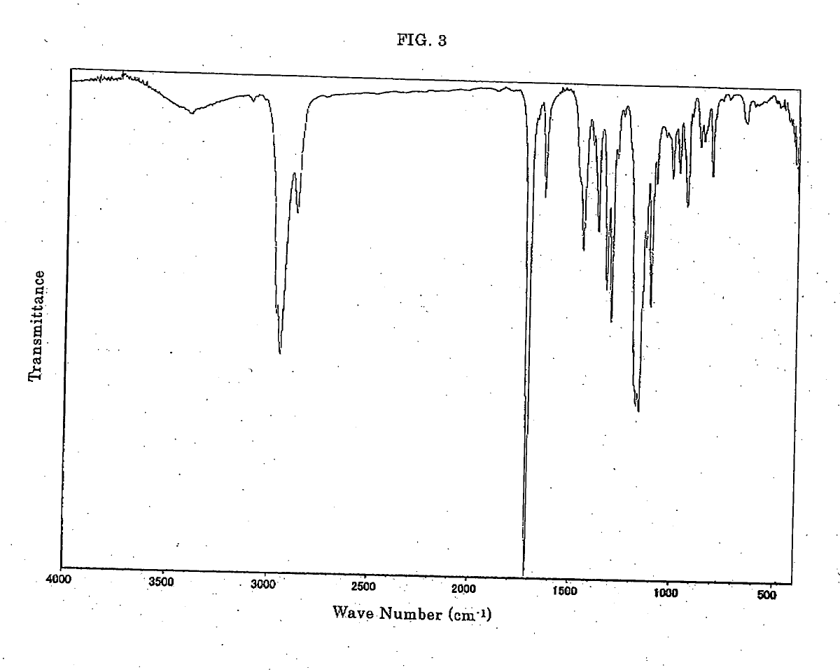

Infrared spectrum: As shown in FIG. 3, an absorption peak of vC=O was observed at 1713 cm⁻¹.

Proton nuclear magnetic resonance spectra (solvent: CDCl₃, 400 MHz):

TABLE 3

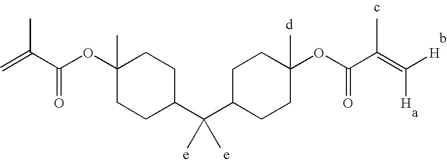

| Assignment | δ (ppm) | Signal | Number of Protons |
|---|---|---|---|
| a | 5.98, 6.02 | s + s | 2 |
| b | 5.42, 5.45 | s + s | 2 |
| c | 1.82, 1.85 | s + s | 6 |
| d | 1.45 | s | 6 |
| e | 0.65 | s | 6 |

### Example 4

### Synthesis of 4,4′-dimethacryloyloxy-4,4′-dimethylbicyclohexyl

Under an atmosphere of argon, a suspension of 5.0 g of 4,4′-dioxobicyclohexyl in 150 mL of anhydrous tetrahydrofuran was placed in a reaction vessel. 18.0 mL of tetrahydrofuran solution of 3.0 mol/L concentration of methylmagnesium chloride was added dropwise from a dropping funnel into the reaction vessel while the resulting reaction mixture was stirred and kept at a temperature of 40° C. or less. Then, the dropping funnel was washed with 25 mL of anhydrous tetrahydrofuran and the wash was also added dropwise from the dropping funnel to the reaction mixture. Thereafter, another 100 mL of anhydrous tetrahydrofuran was added and the reaction mixture was stirred for one hour.

Subsequently, 7.40 g of methacryloyl chloride was added dropwise to the reaction mixture with stirring under an atmosphere of argon, and then the reaction mixture was stirred for 17 hours at room temperature. The resulting reaction mixture was poured into a saturated aqueous solution of ammonium chloride, followed by extracting with ethyl acetate. The thus obtained organic layer was washed with saturated aqueous solution of sodium bicarbonate and water in this order, and was then subjected to concentration under reduced pressure. The thus obtained oily material was purified by silica gel column chromatography (using an eluate of 10% ethyl acetate/hexane), thereby providing 1.6 g of 4,4′-dimethacryloyloxy-4,4′-dimethyl-bicyclohexyl as white solid. The yield was found to be 18 mol %.

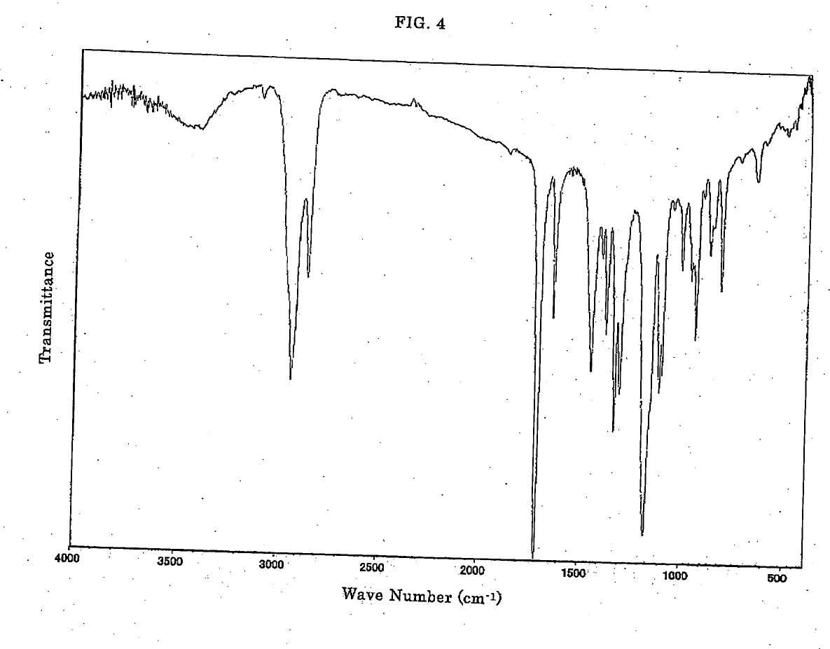

Infrared spectrum: As shown in FIG. 4, an absorption peak of vC=O was observed at 1712 cm⁻¹.

Proton nuclear magnetic resonance spectra (solvent: CDCl₃, 400 MHz):

TABLE 4

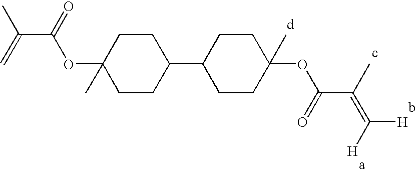

| Assignment | δ (ppm) | Signal | Number of Protons |
|---|---|---|---|
| a | 5.98, 6.02 | s + s | 2 |
| b | 5.42, 5.45 | s + s | 2 |
| c | 1.82, 1.85 | s + s | 6 |
| d | 1.45 | s | 6 |

Examples of Production of (Meth)acrylates

### Example 5

### Synthesis of 1,3,3,5-tetramethylcyclohexyl Methacrylate

115 mL of tetrahydrofuran solution of 3.0 mol/L concentration of methylmagnesium chloride was added into a reaction vessel in which 300 mL of anhydrous tetrahydrofuran had been placed under an atmosphere of argon, and then 40 g of 3,3,5-trimethylcyclohexanone was added with stirring while the mixture in the reaction vessel was kept at a temperature of 50° C., followed by stirring for another one hour at the same temperature with stirring. Then, 120 g of vinyl methacrylate was added drop wise into the reaction vessel, and then 220 g of N, N,N′,N′-tetramethylethylene diamine, and the reaction was carried out with stirring for 17 hours.

After the reaction, the resulting reaction mixture was concentrated under reduced pressure to about half in volume, and the resulting liquid concentrate was extracted with hexane. The thus obtained organic layer which contained the desired product was filtered and washed with water, and was again concentrated under reduced pressure. The obtained concentrate was subjected to sublimation purification in vacuo, thereby providing 12.3 g of 1,3,3,5-tetramethylcyclohexyl methacrylate as colorless oil. The yield was found to be 19.1 mol % as determined by gas chromatography.

The infrared spectrum and the proton nuclear magnetic resonance spectrum were found to be the same as the product obtained in Example 1.

### Example 6

2.5 mL of tetrahydrofuran solution of 3.0 mol/L concentration of methylmagnesium chloride was added to 10 mL of anhydrous tetrahydrofuran in a reaction vessel under an atmosphere of argon, and then 1.0 g of 3,3,5-trimethylcyclohexanone was added with stirring while the mixture in the reaction vessel was kept at a temperature of 50° C. or less, followed by stirring for another one hour at the same temperature with stirring. Then, 2.80 g of vinyl methacrylate was added drop wise into the reaction vessel, and then 4.64 g of N,N,N′,N′-tetramethylethylene diamine, and the reaction was carried out with stirring for 17 hours at room temperature.

After the reaction, an aqueous saturated solution of ammonium chloride was added to the resulting reaction mixture, and the desired product was extracted with ethyl acetate. The obtained organic layer was subjected to gas chromatographic analysis. As a result, the yield of 1,3,3,5-tetramethylcyclohexyl methacrylate was found to be 37.4 mol %.

### Example 7

2.5 mL of tetrahydrofuran solution of 3.0 mol/L concentration of methylmagnesium chloride was added to 10 mL of anhydrous tetrahydrofuran in a reaction vessel under an atmosphere of argon, and then 1.0 g of 3,3,5-trimethylcyclohexanone was added with stirring while the mixture in the reaction vessel was kept at a temperature of 50° C. or less, followed by stirring for another one hour at the same temperature with stirring. Then, 1.20 g of phenyl methacrylate was added drop wise into the reaction vessel, and then 1.74 g of N,N,N′,N′-tetramethylethylene diamine, and the reaction was carried out with stirring for 24 hours at a temperature of 60° C.

After the reaction, an aqueous saturated solution of ammonium chloride was added to the resulting reaction mixture, and the desired product was extracted with ethyl acetate. The obtained organic layer was subjected to gas chromatographic analysis. As a result, the yield of 1,3,3,5-tetramethylcyclohexyl methacrylate was found to be 18.0 mol %.

### Comparative Example 1

In place of phenyl methacrylate, 0.79 g of methyl methacrylate was added dropwise, and then 1.74 g of N,N,N′,N′-tetramethylethylene diamine was added dropwise, and the reaction was carried out for 17 hours, and otherwise in the same manner as in Example 7, an organic layer was obtained. As a result of gas chromatographic analysis of the organic layer, the yield of the desired 1,3,3,5-tetramethylcyclohexyl methacrylate was found to be less than 1 mol %.

### Comparative Example 2

After the dropwise addition of phenyl methacrylate, N,N,N′,N′-tetramethylethylene diamine was not added, and otherwise the reaction was carried out in the same manner as in Example 7, an organic layer was obtained. As a result of gas chromatographic analysis of the organic layer, the yield of the desired 1,3,3,5-tetramethylcyclohexyl methacrylate was found to be less than 1 mol %.

1. A (meth)acrtyte represented by the general formula (I) 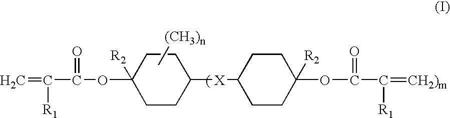
wherein X is a single bond or an alkylidene group having the general formula (II) 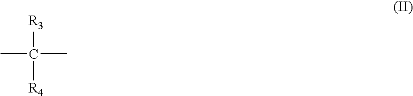
wherein R₃ and R₄ are independently hydrogen atoms or alkyl groups having 1 to 4 carbon atoms, R₁ is a hydrogen atom or a methyl group, R₂ is an alkyl group having 1 to 4 carbon atoms, m is 0 or 1, and when m is 0, n is 3, and when m is 1, n is 0.

2. A 1-Alkylcyclohexyl (meth)acrylate represented by the general formula (Ia) 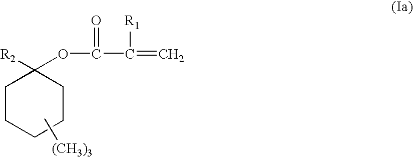
wherein R₁ is a hydrogen atom or a methyl group, and R₂ is an alkyl group having 1 to 4 carbon atoms

3. 1,3,3,5-Tetramethylcyclohexyl (meth)acrylate.

4. A di(meth)acrylate represented by the general formula (Ib) 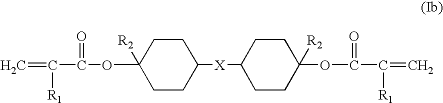
wherein X is a single bond or an alkylidene group having the general formula (II) 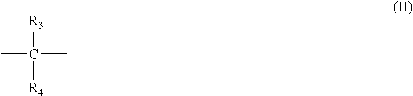
wherein R₃ and R₄ are independently hydrogen atoms or alkyl groups having 1 to 4 carbon atoms, R₁ is a hydrogen atom or a methyl group, and R₂ is an alkyl group having 1 to 4 carbon atoms.

5. 4,4′-dimethacryloyloxy-4,4′-dimethylbicyclohexyl.

6. 2,2-bis(4-methacryloyloxy-4-methylcyclohexyl)propane.

7. A method of producing a cycloaliphatic tertiary (meth)acrylate which comprises reacting a cycloaliphatic ketone with an organomagnesium halide and a (meth)acrylic acid ester represented by the general formula (III) 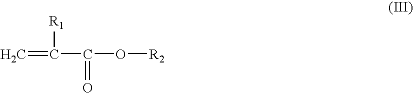
wherein R₁ is a hydrogen atom or a methyl group, and R₂ is an aryl group or a vinyl group, in the presence of an amine.

8. A method of a cycloaliphatic tertiary (meth)acrylate as claimed in claim 7, in which the organomagnesium halide is an alkylmagnesium halide or a phenylmagnesium halide.

9. A method of a cycloaliphatic tertiary (meth)acrylate as claimed in claim 7, in which the amine is an aliphatic tertiary diamine.

10. A method of a cycloaliphatic tertiary (meth)acrylate as claimed in claim 7, in which the (meth)acrylic acid ester is phenyl (meth)acrylate.

11. A method of a cycloaliphatic tertiary (meth)acrylate as claimed in claim 7, in which a cycloaliphatic ketone and an organomagnesium halide are reacted together in a solvent to generate a tertiary cycloaliphatic magnesium halide alkoxide, and then either after the addition of the (meth)acrylic acid ester, an amine is added to the resulting reaction mixture, or both the (meth)acrylic acid ester and the amine are added simultaneously to the resulting reaction mixture, or after the addition of the amine, the (meth)acrylic acid ester is added to the resulting reaction mixture, so as to react the tertiary cycloaliphatic magnesium halide alkoxide and the (meth)acrylic acid ester in the presence of the amine, and then the desired tertiary cycloaliphatic (meth)acrylate is separated from the resulting reaction mixture.
